(19) United States
(12) Patent Application Publication
(10) Pub. No.: US 2001/0010806 A1
(43) Pub. Date: Aug. 2, 2001

(54) Deodorizing apparatus for shoes

(76) Inventors: Toshio Kanazawa, Kasukabe-Shi (JP); Satoshi Watanabe, Saitama (JP); Hiroyoshi Sawada, Saitama (JP); Yasuhiro Tomita, Saitama (JP)

Correspondence Address: Thomas R. Morrison, Esq., MORRISON LAW FIRM, 145 North Fifth Avenue, Mount Vernon, NY, 10550, US

(21) Appl. No.: 09/772,243

(22) Filed: Jan. 29, 2001

(30) Foreign Application Priority Data
Jan. 31, 2000 (JP) 2000-21421
Dec. 5, 2000 (JP) 2000-369437

(51) Int. Cl. A61L 2/10

(52) U.S. Cl. 422/305; 422/24; 422/186.07; 250/454.11

(57) ABSTRACT

A deodorizing apparatus for shoes, said deodorizing apparatus for shoes comprises a pair of leg portions which are to be inserted into the shoes, a joining portion which joins upper ends of the pair of leg portions at a predetermined interval, ozone generating ultraviolet lamps which are respectively received in the pair of leg portions, and a power supply which drives the ultraviolet lamps to light up, wherein each of the leg portions is formed with an ozone emitting portion for emitting ozone generated by the corresponding ultraviolet lamp, and/or an ultraviolet radiation emitting portion for emitting ultraviolet radiation of a wavelength having an ozone generating action as has been generated by the corresponding ultraviolet lamp.

### BACKGROUND OF THE INVENTION

1. Field of the Invention

This invention relates to a deodorizing apparatus for shoes, and more particularly to a deodorizing apparatus for shoes as can deodorize the interiors of the pair of shoes with ozone at one time.

2. Description of the Prior Art

The interior of a shoe is a place where a bad smell is prone to arise due to the sweat of a foot or the propagation of bacilli. Heretofore, a method for removing such bad smells of shoes has been one wherein an ozone generator is mounted in a shoe cupboard, and ozone is generated by the ozone generator, thereby to deodorize the shoes accommodated in the shoe cupboard.

There has also been a method wherein the bad smell of a shoe is removed in such a way that an insole containing the activated carbon of coconut husk or the like adsorbent is inserted in the shoe.

The method wherein the ozone generator is mounted in the shoe cupboard, however, has been incapable of effectively deodorizing those interiors of the shoes which are the origins of the bad smells, though it can really remove the bad smells arising inside the shoe cupboard itself and outside the shoes.

On the other hand, the method wherein the insole containing the adsorbent is inserted in the shoe can remove the bad smell arising inside the shoe, unlike the ozone generation method. Since, however, bad smell components arise successively inside the shoe being often used, the insole has become ineffective soon and has required frequent replacements.

### SUMMARY OF THE INVENTION

This invention has been made in view of the above problems of the prior art, and has for its object to realize a deodorizing apparatus for shoes as can effectively deodorize the interiors of the pair of shoes at one time.

In order to accomplish the object, a deodorizing apparatus for shoes according to this invention comprises a pair of leg portions which are to be inserted into the shoes, a joining portion which joins upper ends of the pair of leg portions at a predetermined interval, ozone generating ultraviolet lamps which are respectively received in the pair of leg portions, and a power supply which drives the ultraviolet lamps to light up, wherein each of the leg portions is formed with an ozone emitting portion for emitting ozone generated by the corresponding ultraviolet lamp, and/or an ultraviolet radiation emitting portion for emitting ultraviolet radiation of a wavelength having an ozone generating action as has been generated by the corresponding ultraviolet lamp.

With the shoe deodorizing apparatus of the present invention, the leg portions can be respectively inserted into the interiors of the right and left shoes after the pair of shoes have been placed astride of the joining portion. In this state, the ultraviolet lamps in the leg portions are driven to light up, and the ozone and/or the ultraviolet radiation of the wavelength having the ozone generating action as have been generated by the ultraviolet lamps are/is emitted from the ozone emitting portion and/or the ultraviolet radiation emitting portion. Thus, the interiors of the pair of shoes can be deodorized at one time by the ozone emitted from the ozone emitting portion and/or ozone generated by the ultraviolet radiation emitted from the ultraviolet radiation emitting portion.

The ozone emitting portion and/or the ultraviolet radiation emitting portion correspond(s) to, for example, an opening formed in the leg portion. When the opening is formed in the leg portion in this manner, the ozone and/or the ultraviolet radiation of the wavelength having the ozone generating action can be emitted out of the leg portion from the opening.

Alternatively, recesses may well be respectively formed in lower end surfaces of said leg portions of the shoe deodorizing apparatus, each of the recesses receiving the corresponding ultraviolet lamp therein and including the opening for emitting the ozone and/or the ultraviolet radiation of the wavelength having the ozone generating action as have/has been generated by the corresponding ultraviolet lamp.

When the leg portions have been inserted into the interiors of the shoes, the lower end surfaces of the leg portions lie at the deepest positions of the interiors of the shoes. Accordingly, the ozone and/or the ultraviolet radiation of the wavelength having the ozone generating action as have/has been generated by each ultraviolet lamp are/is facilitated diffusing inwards of the corresponding shoe by receiving the ultraviolet lamp in the recess formed in the lower end surface of the leg portion.

In the case of forming the recesses, protrusions should desirably be formed on the lower end surface of each leg portion. When the protrusions are formed on the lower end surface of each leg portion, they abut against a sole even in case of a thin-soled shoe, and a predetermined gap is secured between the lower end surface of the leg portion and the sole. Therefore, the ozone and/or the ultraviolet radiation of the wavelength having the ozone generating action as have/has been generated by the ultraviolet lamp are/is diffused inwards of the shoe from the gap.

### BRIEF DESCRIPTION OF THE DRAWINGS

FIG. 1 is a schematic sectional view of a deodorizing apparatus for shoes according to the present invention;

FIG. 2 is a plan view of the deodorizing apparatus for shoes according to the present invention;

FIG. 3 is a bottom view of the deodorizing apparatus for shoes according to the present invention;

FIG. 4 is a sectional view of an ultraviolet lamp which is used in the deodorizing apparatus for shoes according to the present invention;

FIG. 5 is a front view of the trigger electrode of the ultraviolet lamp;

FIG. 6 is a side view of the trigger electrode of the ultraviolet lamp;

FIG. 7 is a bottom view of the trigger electrode of the ultraviolet lamp;

FIG. 8 is an explanatory view showing a state where the gastight envelope of the ultraviolet lamp is inserted into the trigger electrode thereof;

FIG. 9 is a front view of the ultraviolet lamp;

FIG. 10 is a side view of the ultraviolet lamp;

FIG. 11 is a sectional view taken along line A-A indicated in FIG. 9;

FIG. 12 is an explanatory view showing the state of use of the shoe deodorizing apparatus of the present invention; and

FIG. 13 is a block diagram showing the circuit arrangement of the shoe deodorizing apparatus of the present invention.

### DETAILED DESCRIPTION OF THE PREFERRED EMBODIMENTS

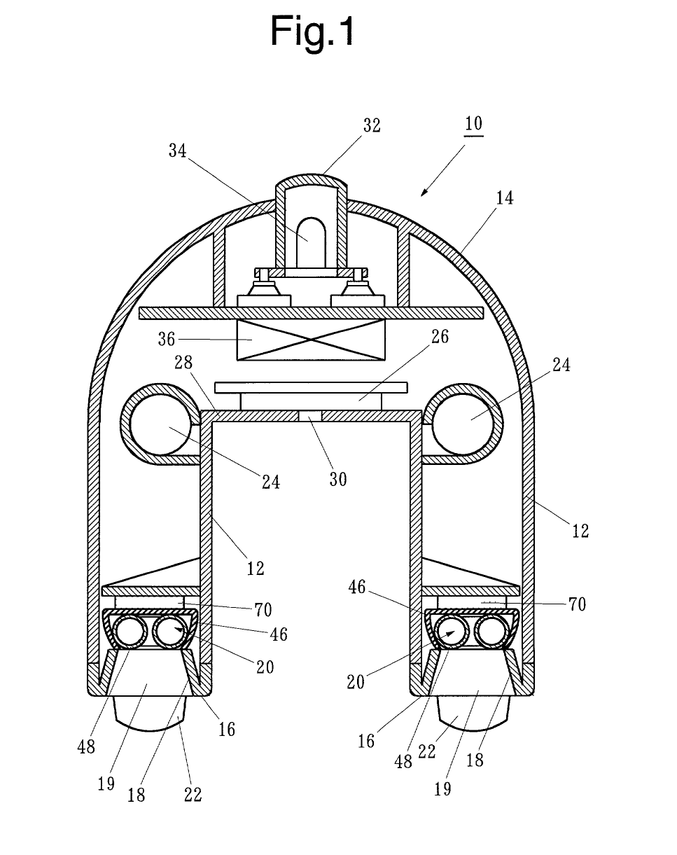

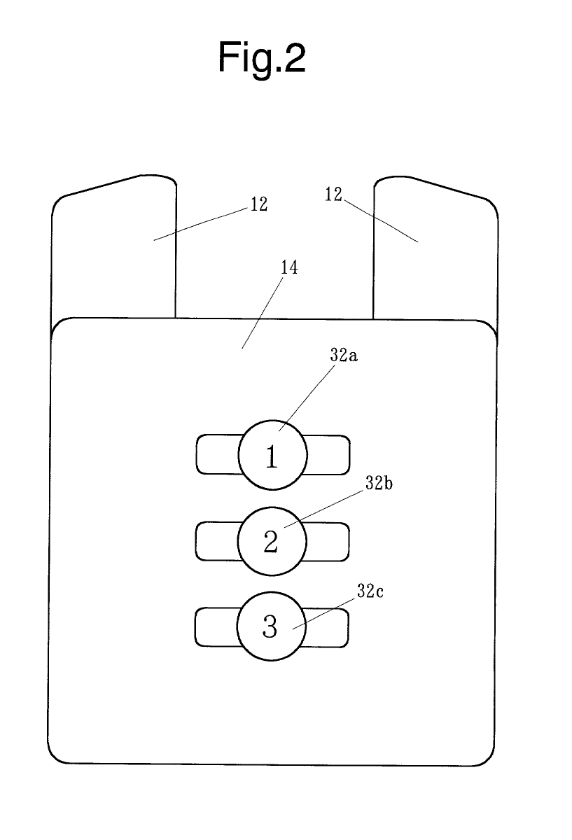

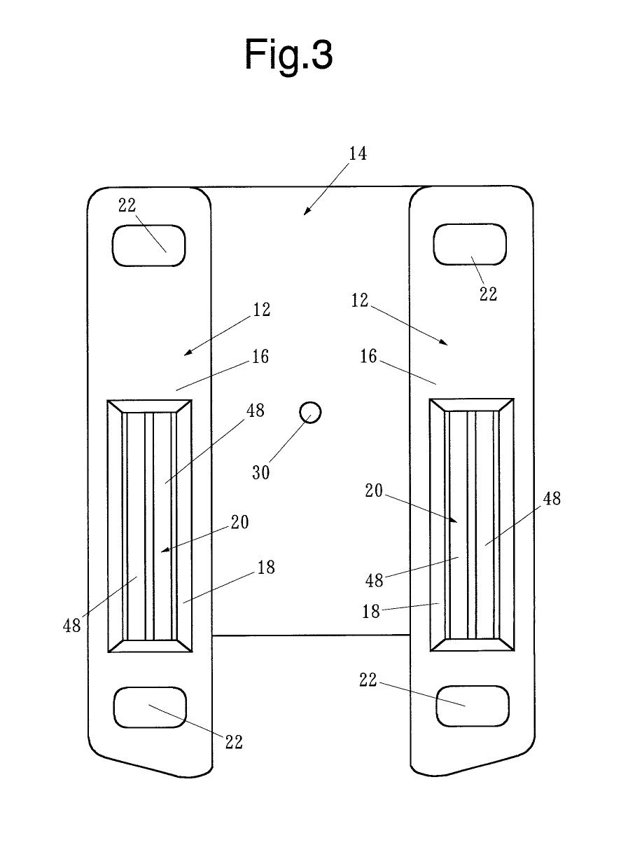

Now, an embodiment of a deodorizing apparatus for shoes according to the present invention will be described with reference to the drawings. FIG. 1 is a schematic sectional view of a deodorizing apparatus for shoes 10 according to the present invention, FIG. 2 is a plan view thereof, and FIG. 3 is a bottom view thereof.

As shown in the figures, the shoe deodorizing apparatus 10 of the present invention includes a pair of tubular leg portions 12, 12 to be inserted into the respective shoes, each of which is substantially in the shape of a rectangular parallelepiped, and a substantially semicylindrical joining portion 14 which joins the upper ends of the pair of tubular leg portions 12, 12 at a predetermined interval.

The lower end surfaces 16 of the pair of leg portions 12, 12 are respectively formed with recesses 18, in which ultraviolet lamps 20 are respectively received.

In addition, protrusions 22 are respectively formed at fore and hind corners in the lower end surface 16 of each leg portion 12. Thus, the shoe deodorizing apparatus 10 of the present invention can be self-supported by the protrusions 22 totaling four, which are formed at the fore and hind corners of the pair of leg portions 12, 12 (as best shown in FIG. 3).

Referring to FIG. 1, numerals 24, 24 designate batteries. The shoe deodorizing apparatus 10 of the present invention can be operated by a total battery voltage of 3 (V) which is produced by the two alkaline batteries of “AAA” size 24, 24. Besides, numeral 26 designates a buzzer for sounding the start and end of the operation of the shoe deodorizing apparatus 10. A sound emitting aperture 30 is formed in that bottom wall 28 of the joining portion 14 on which the buzzer 26 is put. Further, numeral 32 designates an actuating switch. As shown in FIG. 2, such switches 32 in the number of three (a switch 32a with numeral 1 indicated, a switch 32b with numeral 2 indicated, and a switch 32c with numeral 3 indicated) are disposed on the upside of the joining portion 14. An LED (light emitting diode) 34 is built in each switch 32, and it is adapted to blink for a predetermined time in interlocking with the depressed movement of the corresponding switch 32.

Shown at numeral 36 is a drive unit which includes a timer circuit 38 to be explained later, for controlling the lighting-up time of each ultraviolet lamp 20 and the sounding time of the buzzer 26, and an inverter circuit 40 to be explained later, for controlling the lighting-up drive of each ultraviolet lamp 20.

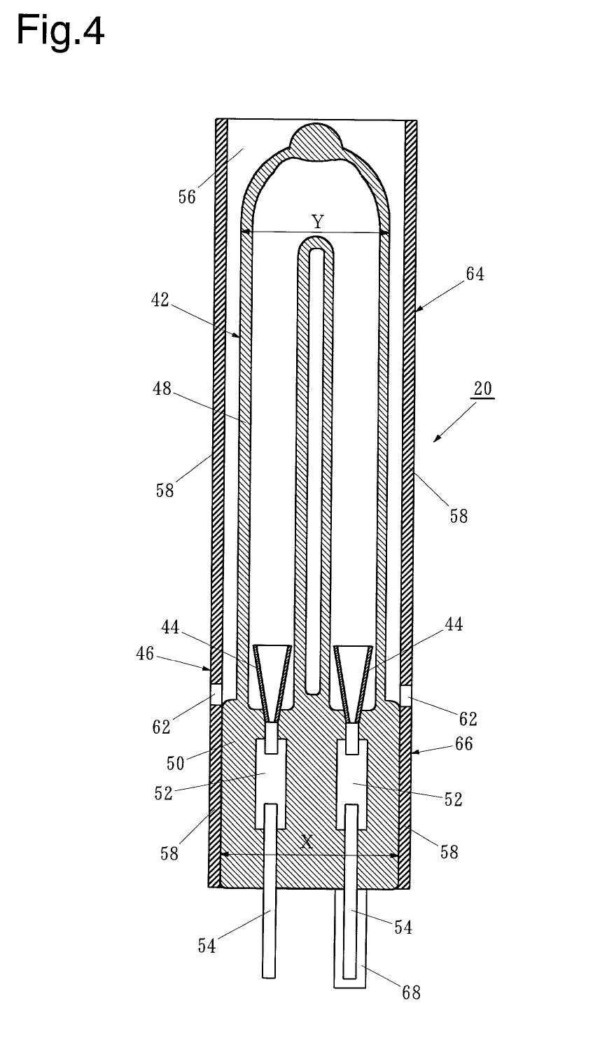

As shown in FIG. 4, the ultraviolet lamp 20 includes a gastight envelope 42 which is formed by bending a slender glass pipe substantially in the shape of letter U and then melting and sealing the openings of both the ends thereof, a pair of discharging electrodes 44, 44 which are respectively arranged in the vicinities of both the end sealed parts of the gastight envelope 42, and a trigger electrode 46 which is arranged around the gastight envelope 42.

The gastight envelope 42 is made of silica glass which has the property of transmitting ultraviolet radiation at wavelengths of at least 180 (nm), and it includes a substantially U-shaped tube portion 48 and a sealing portion 50. Incidentally, since the sealing portion 50 is formed by melting both the end opening parts of the tube portion 48 and thereafter squeeze-molding the molten parts, the lateral width X of this sealing portion 50 is greater than that Y of the tube portion 48.

The tube portion 48 of the gastight envelope 42 is filled up with, for example, an ultraviolet emission gas in which argon and mercury are mixed, or an ultraviolet emission gas which consists principally of xenon, as a discharge gas for generating the ultraviolet radiation.

Each of the discharging electrodes 44, 44 is made of a funnel-shaped member of tungsten, the distal end of which is exposed inside the tube portion 48 of the gastight envelope 42 and the base end of which is welded to a molybdenum foil 52 buried in the sealing portion 50 of the gastight envelope 42. One end of the corresponding one of lead terminals 54, 54 is also connected to the foil 52. In addition, the other end of each of the lead terminals 54, 54 is led out of the gastight envelope 42.

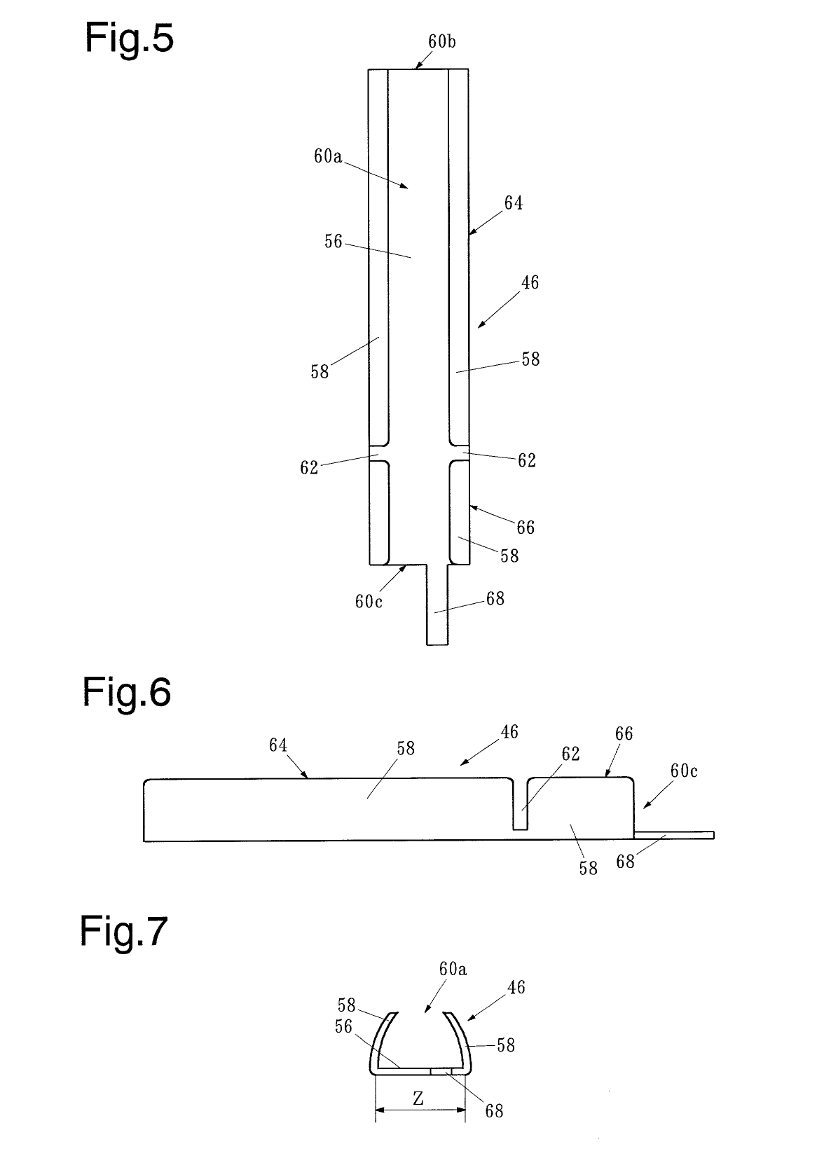

The trigger electrode 46 is disposed in order to permit the low drive voltage of the ultraviolet lamp 20, and it is made of a stainless steel plate having a high reflection efficiency for the ultraviolet radiation. As shown in FIGS. 5 through 7, it includes a flat portion 56, and a pair of crooked portions 58, 58 which are respectively curved substantially in the shapes of circular arcs from both the right and left side edges of the flat portion 56 inward of this flat portion 56. The distal ends of the pair of crooked portions 58, 58 are spaced so as to define an open portion 60a. Also, the upper and lower ends of the trigger electrode 46 define open portions 60b and 60c, respectively.

Notches 62, 62 are respectively provided at the intermediate positions of the pair of crooked portions 58, 58. Referring to FIG. 5, those parts of the crooked portions 58, 58 and the flat portion 56 which lie above the notches 62, 62 constitute a tube portion holder 64 for the gastight envelope 42, and those parts of the crooked portions 58, 58 and the flat portion 56 which lie below the notches 62, 62 constitute a sealing portion holder 66 for the gastight envelope 42. Accordingly, the notches 62, 62 are respectively formed at the positions which correspond to the vicinity of the boundary between the tube portion 48 and sealing portion 50 of the gastight envelope 42 when this gastight envelope 42 is inserted into the trigger electrode 46 (as will be explained later).

Besides, a connection terminal 68 is extended from the lower end of the flat portion 56.

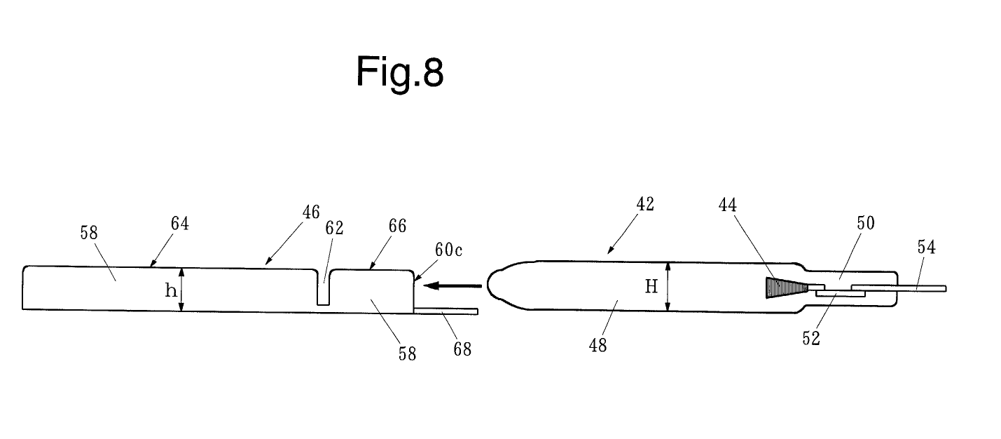

Incidentally, the lateral width Z (FIG. 7) of the flat portion 56 is set somewhat smaller than that X of the sealing portion 50 of the gastight envelope 42, and the height h of the crooked portion 58 is set somewhat smaller than that H of the tube portion 48 of the gastight envelope 42 (as shown in FIG. 8).

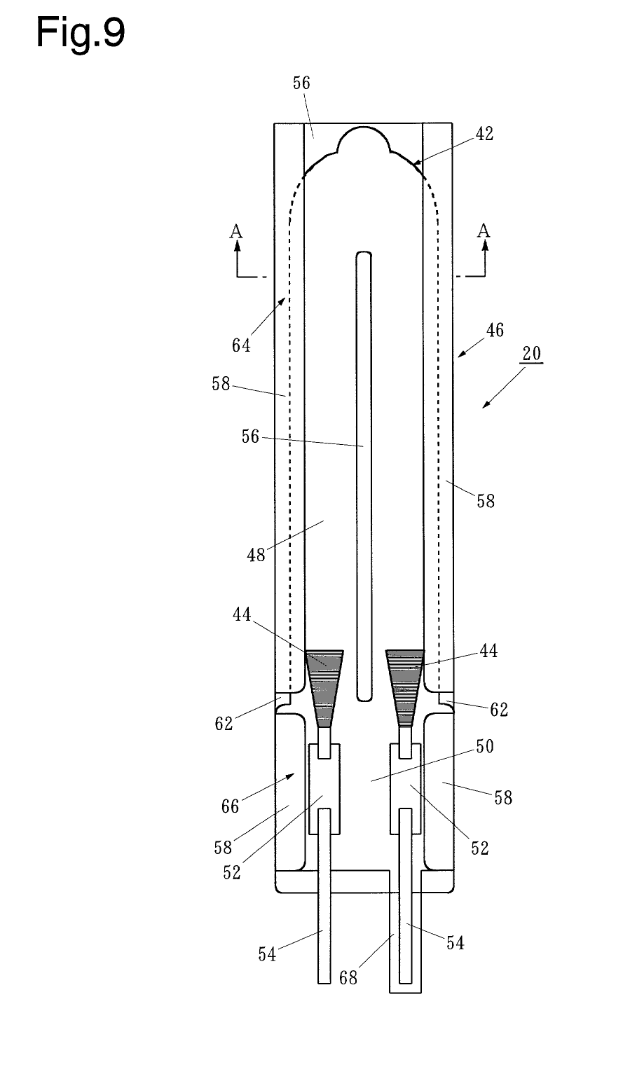

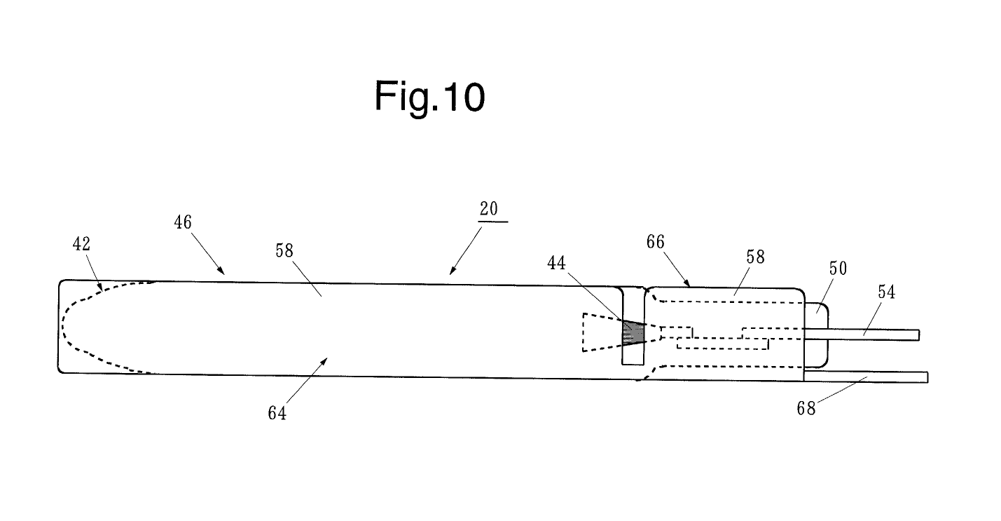

The gastight envelope 42 is inserted from the side of the open portion 60c at the lower end of the trigger electrode 46 having the above construction, as indicated by an arrow in FIG. 8, until the vicinity of the boundary between the tube portion 48 and sealing portion 50 of the gastight envelope 42 is arranged at the position corresponding to the notches 62, 62 of the crooked portions 58, 58 of the trigger electrode 46. In this state, the outer peripheral surface of the gastight envelope 42 is surrounded with the flat portion 56 and crooked portions 58 of the trigger electrode 46, except those parts of the gastight envelope 42 which correspond to the open portion 60a between the distal ends of the crooked portions 58, 58 and the open portions 60b, 60c at the upper and lower ends in the trigger electrode 46 (refer to FIGS. 9 and 10).

Incidentally, as shown in FIGS. 1 and 3, in the state where the ultraviolet lamp 20 is received in the recess 18 of the leg portion 12, those parts of the tube portion 48 which are not surrounded with the flat portion 56 and crooked portions 58, 58 of the trigger electrode 46 are exposed inside the recess 18.

Besides, a cushion member 70 made of foamed urethane is held in close contact with the back surface of the flat portion 56 of the trigger electrode 46, namely, the surface of the flat portion 56 opposite to the surface thereof confronting the tube portion 48 (as shown in FIG. 1). As a result, the shock resistance of the ultraviolet lamp 20 is heightened.

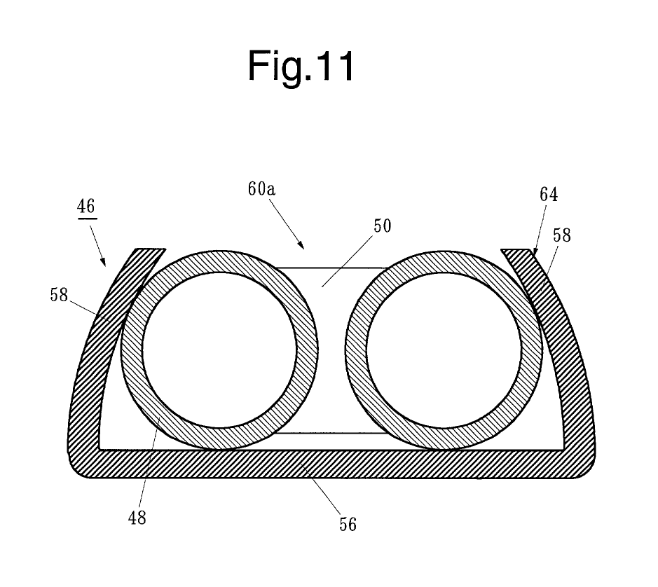

As stated before, the height h of the crooked portion 58 of the trigger electrode 46 is set somewhat smaller than that H of the tube portion 48 of the gastight envelope 42. Therefore, the crooked portions 58, 58 of the trigger electrode 46 come into pressed contact with the tube portion 48 of the gastight envelope 42. As a result, the tube portion 48 is securely held in pressed contact by the crooked portions 58, 58 and flat portion 56 which constitute the tube portion holder 64 of the trigger electrode 46 (refer to FIG. 11).

Moreover, the lateral width Z of the flat portion 56 of the trigger electrode 46 is set somewhat smaller than that X of the sealing portion 50 of the gastight envelope 42. Therefore, the crooked portions 58, 58 of the trigger electrode 46 come into pressed contact with the sealing portion 50 of the gastight envelope 42. As a result, the sealing portion 50 is securely held in pressed contact by the crooked portions 58, 58 which constitute the sealing portion holder 66 of the trigger electrode 46 (refer to FIG. 4).

As stated above, the gastight envelope 42 of the ultraviolet lamp 20 is securely held in pressed contact by the tube portion holder 64 and sealing portion holder 66 of the trigger electrode 46, so that the mechanical strength of the gastight envelope 42 is enhanced. It is accordingly possible to thin the silica glass constructing the gastight envelope 42, with the sufficient mechanical strength ensured, and to increase the quantity of transmission of the ultraviolet radiation through the gastight envelope 42.

Incidentally, the reason why the notches 62, 62 are provided in the respective crooked portions 58, 58 of the trigger electrode 46 at the positions corresponding to the vicinity of the boundary between the tube portion 48 and sealing portion 50 of the gastight envelope 42, is as follows: The lateral width X of the sealing portion 50 of the gastight envelope 42 is greater than that Y of the tube portion 48 thereof. In the absence of the notches 62, 62, therefore, when those parts of the crooked portions 58, 58 which correspond to the sealing portion holder 66 expand outwards along the outer periphery of the sealing portion 50 of the gastight envelope 42, also those parts of the crooked portions 58, 58 which correspond to the tube portion holder 64 will expand outwards substantially equally to the parts of the crooked portions 58, 58 corresponding to the sealing portion holder 66. As a result, the parts of the crooked portions 58, 58 corresponding to the tube portion holder 64 will fail to hold the tube portion 48 of the gastight envelope 42 in pressed contact. In contrast, in the case of providing the notches 62, 62 as in the embodiment, the parts of the crooked portions 58, 58 corresponding to the tube portion holder 64 and those of the crooked portions 58, 58 corresponding to the sealing portion holder 66 fall into a separated state. Therefore, even when the parts of the crooked portions 58, 58 corresponding to the sealing portion holder 66 expand outwards along the outer periphery of the sealing portion 50 of the gastight envelope 42, the parts of the crooked portions 58, 58 corresponding to the tube portion holder 64 do not expand outwards. As a result, the parts of the crooked portions 58, 58 corresponding to the tube portion holder 64 can reliably hold the tube portion 48 of the gastight envelope 42 in pressed contact.

In driving the ultraviolet lamp 20, the connection terminal 68 of the trigger electrode 46 and the mating lead terminal 54 of one discharging electrode 44 are connected, whereupon an A. C. voltage at 50-60 (kHz) is applied to the pair of discharging electrodes 44, 44 and the trigger electrode 46 through the inverter circuit 40 (in FIG. 13). In this case, the voltage of the trigger electrode 46 becomes the same polarity as that of one discharging electrode 44 connected through the connection terminal 68 as well as the mating lead terminal 54, and the opposite polarity to that of the other discharging electrode 44. In this regard, the distance between the trigger electrode 46 and the other discharging electrode 44 of the opposite polarity is smaller than the discharging gap between the pair of discharging electrodes 44, 44. Therefore, when the A. C. voltage is applied, a high electric-field state is first established between the trigger electrode 46 and the other discharging electrode 44, with the result that ions are emitted in large quantities into the tube portion 48 of the gastight envelope 42. Since the large quantities of emitted ions form the origin of the firing across the discharging electrodes 44, 44, the drive of the ultraviolet lamp 20 is permitted even with the low voltage. Consequently, the lifetime of the battery 24 can be lengthened in the shoe deodorizing apparatus 10 of the present invention employing the battery 24 as its power supply.

Incidentally, the ions emitted into the tube portion 48 by the high electric-field state established between the trigger electrode 46 and the other discharging electrode 44 are attracted toward one discharging electrode 44 opposite in polarity to these ions, thereby to proceed inside the tube portion 48. On this occasion, in the structure of the ultraviolet lamp 20, the outer peripheral surface of the tube portion 48 is surrounded with the flat portion 56 and crooked portions 58, 58 of the trigger electrode 46, and the trigger electrode 46 opposite in polarity to the emitted ions is equivalently arranged along the discharging path across the discharging electrodes 44, 44 within the tube portion 48. Therefore, the proceeding of the ions is accelerated to contribute to the low voltage drive of the ultraviolet lamp 20.

By the way, according to an experiment carried out by inventors, when the trigger electrode 46 was adopted for an ultraviolet lamp whose drive voltage was rated at 2.5 (V), the drive voltage could be lowered to 1.7 (V). Therefore, the shoe deodorizing apparatus 10 of the present invention employing such ultraviolet lamps 20, 20 can be operated for a long time by the two alkaline batteries of “AAA” size 24, 24.

When discharge is created across the discharging electrodes 44, 44, ultraviolet radiation at a wavelength of 185 (nm) having an ozone generating action and ultraviolet radiation at a wavelength of 254 (nm) having a sterilizing action are emitted from the ultraviolet emission gas.

The emitted ultraviolet radiations are transmitted through those parts of the tube portion 48 which correspond to the respective open portions 60a and 60b of the trigger electrode 46 and which are not surrounded with the flat portion 56 and crooked portions 58, 58 of the trigger electrode 46. The ultraviolet radiation at the wavelength of 185 (nm) transmitted through the tube portion 48 acts on oxygen contained in the air, thereby to generate ozone. The generated ozone is emitted out of the leg portion 12 from the opening 19 of the recess 18. Besides, part of the ultraviolet radiation at the wavelength of 185 (nm) is emitted out of the leg portion 12 from the opening 19 of the recess 18, and it acts on the air outside the leg portion 12, thereby to generate ozone.

Further, the ultraviolet radiation at the wavelength of 254 (nm) transmitted through the tube portion 48 is emitted out of the leg portion 12 from the opening 19 of the recess 18.

Incidentally, as stated before, the trigger electrode 46 is made of the stainless steel plate which has the high reflection efficiency for the ultraviolet radiation. Therefore, the ultraviolet radiations emitted toward the flat portion 56 and crooked portions 58, 58 of the trigger electrode 46 surrounding the tube portion 48 are reflected from the surfaces of these portions 56 and 58, 58 confronting the tube portion 48, and they are guided toward the parts of the tube portion 48 not surrounded with the flat portion 56 and crooked portions 58, 58 of the trigger electrode 46.

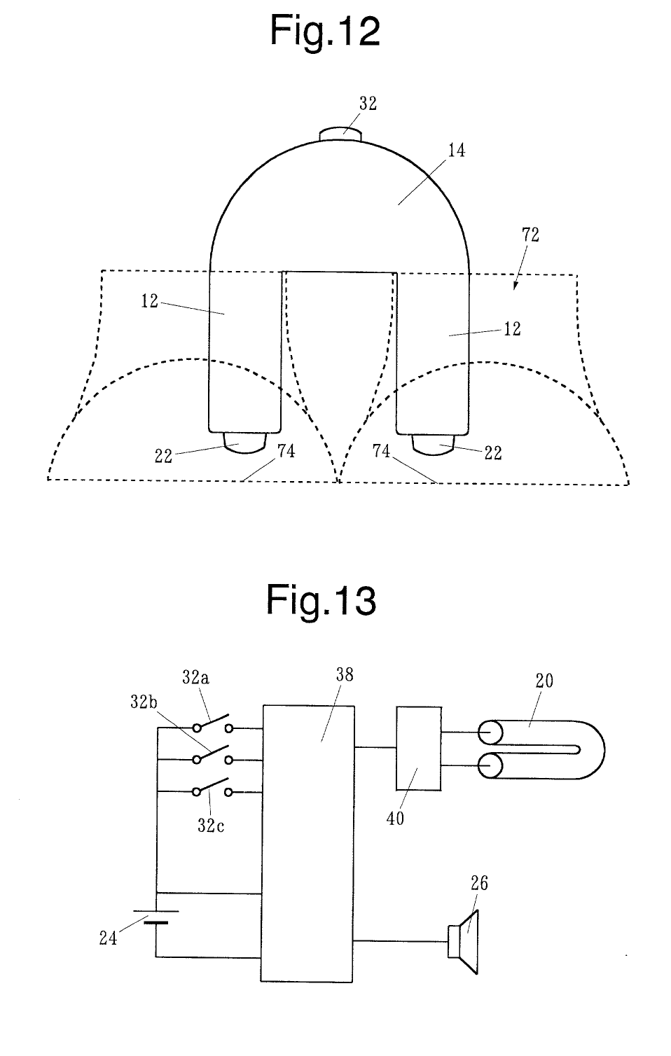

The practicable usage of the shoe deodorizing apparatus 10 according to the present invention having the above construction will be described with reference to FIGS. 12 and 13 below.

First, as shown in FIG. 12, a pair of shoes 72, 72 are placed astride of the joining portion 14 of the shoe deodorizing apparatus 10 of the present invention, and the leg portions 12, 12 are respectively inserted into the interiors of the right and left shoes 72, 72. In this state, the ultraviolet lamps 20, 20 received in the recesses 18, 18 of the leg lower-end surfaces 16, 16 and the soles 74, 74 of the shoes 72, 72 are arranged in opposition with a predetermined spacing held therebetween.

Incidentally, even in case of thin-soled shoes such as high heels, gaps corresponding to the height of the protrusions 22, 22, 22, 22 are secured between the lower end surfaces 16, 16 of the leg portions 12, 12 and the soles 74, 74 for the reason that, as stated before, the shoe deodorizing apparatus 10 is self-supported by the protrusions 22, 22, 22, 22 formed on the lower end surfaces 16, 16 of the leg portions 12, 12.

Next, when the switch 32 disposed on the upside of the joining portion 14 is depressed, the ultraviolet lamps 20, 20 are driven to light up for a predetermined time by the operations of the timer circuit 38 and the inverter circuit 40.

More specifically, when the switch 32a bearing numeral 1 (refer also to FIG. 2) is depressed, the ultraviolet lamps 20, 20 are lit up for 1 minute in total by changing-over the ultraviolet lamp 20 of one leg portion 12 and that 20 of the other leg portion 12 every 30 seconds. When the switch 32b bearing numeral 2 is depressed, the ultraviolet lamps 20, 20 are lit up for 2 minutes in total by changing-over the ultraviolet lamp 20 of one leg portion 12 and that 20 of the other leg portion 12 every 30 seconds, and when the switch 32c bearing numeral 3 is depressed, the ultraviolet lamps 20, 20 are lit up for 3 minutes in total by changing-over the ultraviolet lamp 20 of one leg portion 12 and that 20 of the other leg portion 12 every 30 seconds. Accordingly, the user of the shoe deodorizing apparatus 10 can select any of the three sorts of lighting-up times of the ultraviolet lamps 20, 20 in accordance with the extent of the bad smell of the shoes 72, 72.

Incidentally, the consumption of the batteries 24, 24 can be relieved by alternately driving the ultraviolet lamp 20 of one leg portion 12 and that 20 of the other leg portion 12 to light up as stated above.

The timer circuit 38 is set so as to sound the buzzer 26 for 64 (milliseconds) at the depression of the switch 32, and for 30 (seconds) at the end of the lighting-up of the ultraviolet lamps 20, 20.

Incidentally, the LED 34 built in the switch 32 blinks for indication at intervals of 2 (seconds) in interlocking with the depressed movement of the switch 32.

When the switch 32 is depressed to light up the ultraviolet lamp 20, ozone is generated by ultraviolet radiation at a wavelength of 185 (nm) emitted from the ultraviolet lamp 20, and it is emitted out of the leg portion 12 from the opening 19 of the recess 18. Since the emitted ozone is heavier than the air, it diffuses toward the sole 74, with the result that the interior of the shoe 72 is deodorized and sterilized by the function of the ozone. Besides, part of the ultraviolet radiation at the wavelength of 185 (nm) is emitted out of the leg portion 12 from the opening 19 of the recess 18, and it acts on the air outside the leg portion 12, thereby to generate ozone, by which the interior of the shoe 72 is deodorized and sterilized.

Further, ultraviolet radiation at a wavelength of 254 (nm) having a sterilizing action is emitted from the ultraviolet lamp 20, and it is emitted out of the leg portion 12 from the opening 19 of the recess 18. Since the ultraviolet radiation at the wavelength of 254 (nm) is also emitted and diffused toward the sole 74, the propagation of bacilli forming the origin of the bad smell can be suppressed.

Incidentally, even in the case of using the shoe deodorizing apparatus 10 of the present invention for the thin-soled shoes, the gap corresponding to the height of the protrusions 22 formed on the leg portion 12 is secured between the lower end surface 16 of the leg portion 12 and the sole 74 as explained before, and hence, the ozone and the ultraviolet radiation at the wavelength of 254 (nm) as are generated by the ultraviolet lamp 20 are diffused through the gap.

As thus far described, according to the shoe deodorizing apparatus 10 of the present invention, a pair of shoes 72, 72 are placed astride of a joining portion 14, and leg portions 12, 12 are respectively inserted into the interiors of the right and left shoes 72, 72, whereby the interiors of the pair of shoes 72, 72 can be deodorized at one time owing to the functions of ozone and ultraviolet radiation generated by the ultraviolet lamps 20, 20 received in the leg portions 12, 12.

What is claimed is:

1. A deodorizing apparatus for shoes, comprising a pair of leg portions which are to be inserted into the shoes, a joining portion which joins upper ends of said pair of leg portions at a predetermined interval, ozone generating ultraviolet lamps which are respectively received in said pair of leg portions, and a power supply which drives said ultraviolet lamps to light up, wherein each of said leg portions is formed with an ozone emitting portion for emitting ozone generated by the corresponding ultraviolet lamp, and/or an ultraviolet radiation emitting portion for emitting ultraviolet radiation of a wavelength having an ozone generating action as has been generated by said corresponding ultraviolet lamp.

2. A deodorizing apparatus for shoes as defined in claim 1, wherein said ozone emitting portion and/or said ultraviolet radiation emitting portion are/is defined by an opening formed in said corresponding leg portion.

3. A deodorizing apparatus for shoes as defined in claim 2, wherein recesses are respectively formed in lower end surfaces of said leg portions, each of said recesses receiving said corresponding ultraviolet lamp therein and including said opening for emitting the ozone and/or the ultraviolet radiation of the wavelength having the ozone generating action as have/has been generated by said corresponding ultraviolet lamp.

4. A deodorizing apparatus for shoes as defined in claim 3, wherein protrusions are formed on the lower end surface of said each leg portion.

5. A deodorizing apparatus for shoes as defined in any of claims 1 through 4, wherein said each ultraviolet lamp comprises a gastight envelope which is made of an ultraviolet radiation transmitting substance, and which includes a tube portion and a sealing portion formed by sealing both end openings of said tube portion, a pair of discharging electrodes which are respectively arranged near both the end sealed parts inside said gastight envelope, an ultraviolet emission gas with which said tube portion of said gastight envelope is filled up, and a trigger electrode which is arranged around said gastight envelope; said trigger electrode includes a tube portion holder which holds said tube portion of said gastight envelope in pressed contact, and a sealing portion holder which holds said sealing portion of said gastight envelope in pressed contact; and a distance between said trigger electrode and each of said discharging electrodes is made smaller than a discharging gap between said pair of discharging electrodes.
